(19) United States
(12) Patent Application Publication
(10) Pub. No.: US 2013/0016807 A1
(43) Pub. Date: Jan. 17, 2013

(54) MAMMOGRAPHY X-RAY DEVICE

(71) Applicants: Jacqueline Kallert, Erlangen (DE); Robert Standar, Pretzfeld (DE)

(72) Inventors: Jacqueline Kallert, Erlangen (DE); Robert Standar, Pretzfeld (DE)

(21) Appl. No.: 13/351,172

(22) Filed: Jan. 16, 2012

(30) Foreign Application Priority Data
Jan. 17, 2011 (DE) 10 2011 002 758.0

(51) Int. Cl. A61B 6/04

(52) U.S. Cl. 378/37

(57) ABSTRACT

A mammography x-ray device includes a compression apparatus with a compression plate that may be displaced along an axis. The compression apparatus has a drive device that may be actuated by an operating element to displace the compression plate.

This application claims the benefit of DE 10 2011 002 758.0, filed on Jan. 17, 2011.

### BACKGROUND

The present embodiments relate to a mammography x-ray device having a compression apparatus with a compression plate that is displaceable along an axis.

Mammography is a procedure for examining a breast using x-rays (e.g., for the early identification of breast cancer).

Mammography x-ray devices essentially include an x-ray radiation source for emitting x-ray beams, a compression apparatus including a compression plate for the breast, and a table. The table, in the case of analog x-ray devices, includes a film holder or cassette holder. In the case of digital x-ray devices, the table includes an electronic sensor. During the examination, the breast is pressed against the table by the compression plate to enhance image quality and reduce radiation exposure.

A conventional mammography x-ray device is known from DE 10 2008 011 154 A1. With such devices, the compression plate may be displaced manually in a horizontal direction. Three fixed locking positions may, for example, be provided to allow adjustment to different examination objects.

Operation (e.g., the setting of the x-ray dose and other examination parameters) is performed either directly at the mammography x-ray device or at an operating point (e.g., a work station). Before the mammography examination, a position of the compression plate is input manually at an operating element. It is therefore possible for the compression plate to be displaced to the right, for example, but for the operator inadvertently to input that the compression plate is on the left side. This results in incorrect exposure, providing that the image recording is unusable and has to be repeated. As a result, the patient is exposed unnecessarily to an increased radiation dose.

### SUMMARY AND DESCRIPTION

The present embodiments may obviate one or more of the drawbacks or limitations in the related art. For example, a mammography x-ray device that avoids incorrect exposures may be provided.

A compression apparatus of a mammography x-ray device includes a drive device that may be actuated by an operating element to displace a compression plate.

Inadvertent incorrect inputs and incorrect operations may be avoided when setting the mammography x-ray device by controlling the displacement of the drive plate using the operating element. A drive device that may be actuated by the operating element and moves the compression plate to a desired position serves for this purpose. This dispenses with the need for the step of displacing the compression plate manually; with the mammography x-ray device of the present embodiments, the correct setting is found automatically by an input of the user at the operating element.

In one embodiment, the operating element may be integrated in the mammography x-ray device, or the operating element may be a component of a separate input and operating apparatus. The mammography x-ray device may have the operating element on one side; alternatively, the input and operating apparatus may, for example, be configured as a work station that is configured as separate from the mammography x-ray device and connected to the mammography x-ray device.

In one embodiment of the mammography x-ray device, the drive device includes an electric motor (e.g., a geared motor). The geared motor allows precise displacement and positioning of the compression plate based on the input of the user.

In one embodiment, the electric motor (e.g., the geared motor) has a pinion engaging with a rack. The rack converts a rotational movement of the electric motor to a linear movement, so that the compression plate may be displaced along an axis.

The electric motor (e.g., the geared motor) may be disposed on a vertically adjustable holder, and the rack may be disposed on the compression plate or on a component connected to the compression plate. The electric motor may also be disposed on the compression plate, and the rack may be disposed on the holder.

To achieve a precisely defined displacement movement, the rack may be disposed on a guide rail connected to the compression plate. The guide rail allows the compression plate to be displaced laterally in relation to the holder. The guide rail may engage behind the compression plate, thus holding and guiding the compression plate reliably. In one embodiment, the guide rail connected to the compression plate may be supported on the holder using a sliding guide.

A high level of reliability results with the mammography x-ray device of the present embodiments if the compression apparatus has at least one sensor device for detecting the position of the compression plate relative to the holder. The sensor device allows regulation in that the position of the compression plate detected by the sensor device is taken into account when controlling the displacement movement of the compression plate.

The sensor device may include at least one light barrier (e.g., a forked light barrier) to detect the position of the compression plate (e.g., to detect recesses in a component connected to the compression plate). The recesses are disposed such that the recesses are assigned to certain positions of the compression plate, the reaching of which prompts the sensor device to deliver a signal or a modified signal.

The mammography x-ray device may also include a control apparatus or may be coupled to a control apparatus that is configured to control the drive device based on the signal delivered by the sensor device. This allows the adjusting movement of the compression plate to be monitored and the operating element to be coupled to the drive device, thereby providing reliable and error-free operation of the mammography x-ray device.

### BRIEF DESCRIPTION OF THE DRAWINGS

FIG. 1 shows a perspective view of one embodiment of a mammography x-ray device;

FIG. 2 shows a detail of one embodiment of the mammography x-ray device in the region of a drive device;

FIG. 3 shows a detail of one embodiment of the mammography x-ray device in the region of a guide;

FIG. 4 shows a perspective view of one embodiment of a guide rail;

FIG. 5 shows a detail of one embodiment of the mammography x-ray device in the region of the guide rail; and

FIG. 6 shows a perspective view of one embodiment of the compression plate.

### DETAILED DESCRIPTION OF THE DRAWINGS

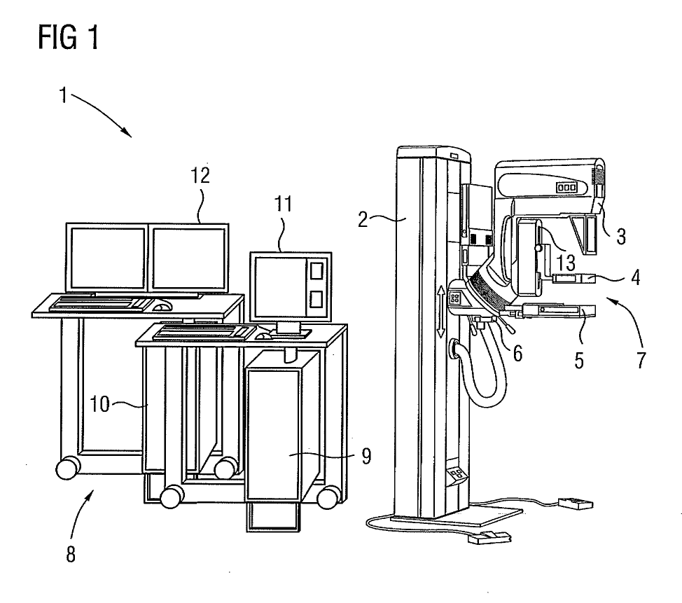

One embodiment of a mammography x-ray device 1 is shown in FIG. 1. The mammography x-ray device 1 includes a stand 2, on which x-ray recording equipment is disposed in a vertically adjustable manner. The mammography x-ray device includes a beam source 3, a vertically adjustable compression plate 4 and a table 5. In the case of analog x-ray devices, the table 5 includes a film holder or a cassette holder; in the case of digital x-ray devices, the table 5 includes an electronic sensor. The beam source 3, the compression plate 4 and the table 5 are disposed on a support arm 6. A height of the support arm 6 may be adjusted relative to the stand 2. The support arm 6 also has an articulation, such that the recording equipment may be turned.

While x-ray images are being recorded, a breast to be examined is compressed, between the compression plate 4 and the table 5. In addition to the compression plate 4, a compression apparatus 7 also includes a drive device (not shown in FIG. 1) to displace the compression plate 4.

The mammography x-ray device 1 is operated by way of an operating element 8. In the embodiment shown in FIG. 1, this includes work stations 9, 10 with assigned screens 11, 12. Settings used for the x-ray image recording (e.g., compression settings, a kV value setting, and/or settings of other parameters influencing image recording) are performed at the work stations 9, 10. Predefined image recording programs may also be selected. These settings are performed at the work station 9. When the images have been recorded, the images may be evaluated at the work station 10.

As shown in FIG. 1, the compression plate 4 may be displaced vertically along a guide 13. In order to achieve the compression used for x-ray image recording, the compression plate 4 is moved in a motorized fashion in the direction of the table 5.

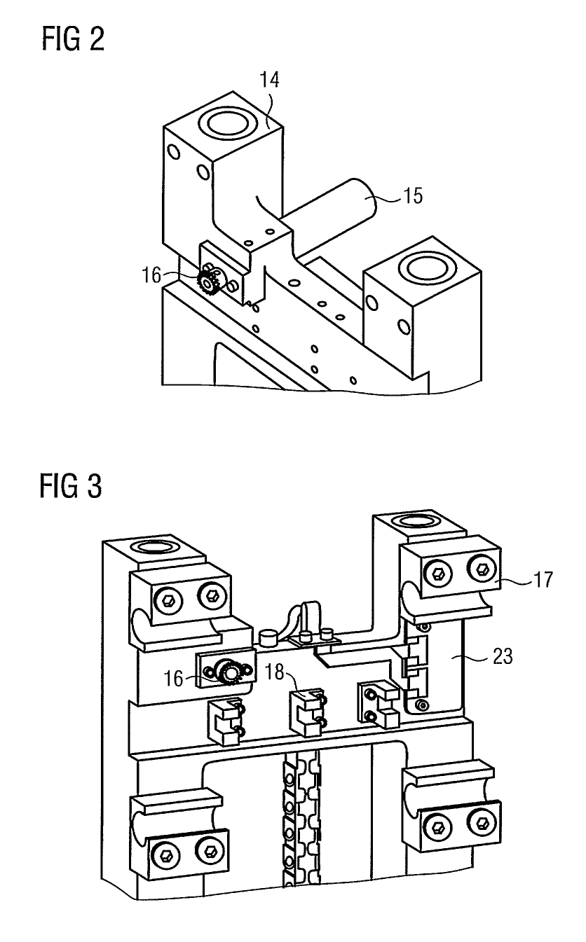

In addition to this vertical adjustability, the compression plate 4 may also be displaced horizontally. FIG. 2 shows a detail of the mammography x-ray device 1 without the compression plate. Vertical adjustment takes place using a holder that is, for example, configured as a lifting carriage 14. The holder has two tubular segments (e.g., vertical segments) that may be displaced along a vertical guide. A drive device configured as an electric motor 15 is disposed on a central segment between the two tubular segments, and a shaft of the electric motor 15 is provided with a pinion 16.

FIG. 3 shows the lifting carriage 14 with further components of the mammography x-ray device 1. The vertical segments of the lifting carriage 14 include a total of four housing bearings 17 that support and guide a guide rail. A number of sensor devices configured as, for example, forked light barriers 18 are disposed in a central region of the lifting carriage 14. The forked light barriers 18 may be used to detect a displacement position of the guide rail.

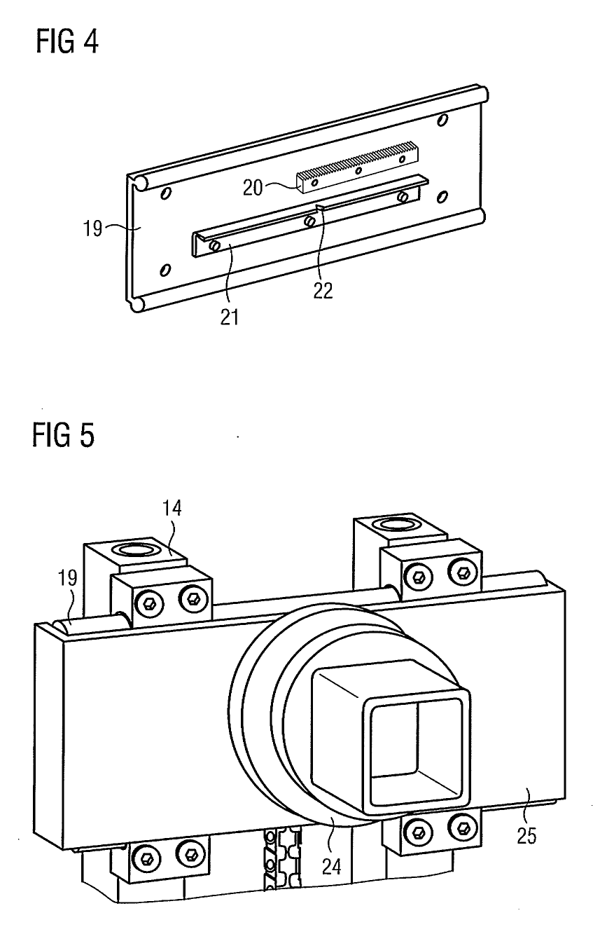

FIG. 4 shows one embodiment of a guide rail 19. The guide rail 19 has a rectangular basic form. A top edge and a bottom edge of the guide rail 19 have an essentially circular cross section that is matched to an internal cross section of one of the housing bearings 17 of the lifting carriage 14. On a side facing the lifting carriage 14, the guide rail 19 has a rack 20 that engages with the pinion 16 disposed on the lifting carriage 14. The guide rail 19 also has a notched plate 21 that is disposed parallel to the rack 20. When the guide rail 19 is driven by the electric motor 15, a rotational movement of the electric motor 15 is converted by the rack 20 to a linear movement. This moves the notched plate 21 along the forked light barriers 18. When a notch 22 passes one of the forked light barriers 18, a signal is triggered or a signal is modified, allowing a certain relative position of the guide rail 19 in relation to the lifting carriage 14 to be detected. This signal generated by the forked light barrier 18 is transmitted to a control facility (e.g., a control system) disposed on a printed circuit board 23 and connected to the work station 9. The displacement and positioning of the guide rail 19 may therefore be controlled from the work station 9 using the electric motor 15.

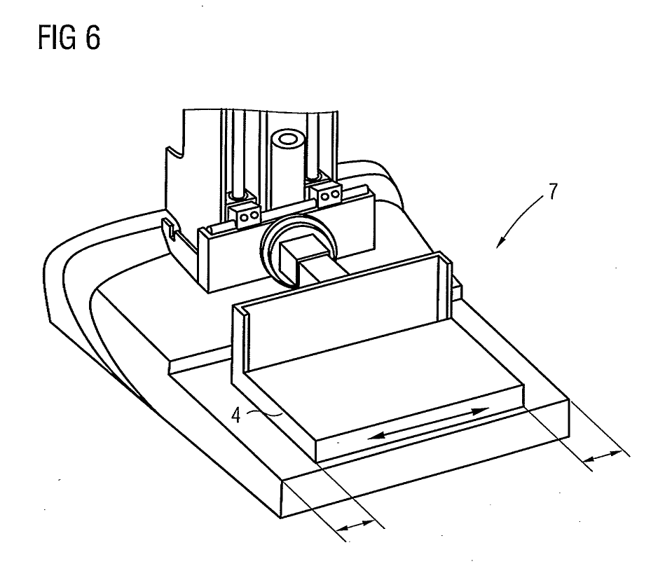

FIG. 5 shows a similar view of a detail of the mammography x-ray device 1 to the view shown in FIG. 3, but with an unlocking mechanism 24 fitted. The unlocking mechanism 24 locks the compression plate 4 once a desired position has been reached, so that no components of the mammography x-ray device 1 move when the image is recorded. As shown in FIG. 5, the guide rail 19 is enclosed by a locking housing 25.

FIG. 6 shows the compression apparatus 7 with the compression plate 4. The displaceable guide rail 19 allows the compression plate 4 to be displaced horizontally. Two outer positions of the compression plate 4 may be set along a sliding guide, starting from a central position. In the embodiment shown in FIG. 6, the displacement is +−30 mm. The compression plate 4 is displaced using the electric motor 15, the pinion 16 of which engages with the rack 20 and displaces the guide rail 19. The guide rail may be moved along the sliding guide together with the compression plate 4. The movement of the compression plate 4 is controlled based on the signal of the forked light barriers 18, which are positioned on the lifting carriage 14. The movement of the notched plate 21 causes the forked light barriers 18 to receive a signal that is used for position detection. The electric motor 15 is controlled by way of a control apparatus provided on the printed circuit board 23. Only one guide rail is used to allow the horizontal displacement of compression plates 4 of different sizes. The electric motor 15 is controlled by way of suitable software that takes the detected position into account and prevents horizontal movement during compression.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A mammography x-ray device comprising:
a compression apparatus comprising:
a compression plate, the compression plate being displaceable along an axis; and
a drive device that is actuatable by an operating element, such that the compression plate is displaced.

2. The mammography x-ray device as claimed in claim 1, wherein the operating element is integrated in a mammography x-ray system or is a component of an input and operating apparatus separate from the mammography x-ray system.

3. The mammography x-ray device as claimed in claim 1, wherein the drive device comprises an electric motor.

4. The mammography x-ray device as claimed in claim 3, wherein the electric motor has a pinion that engages with a rack.

5. The mammography x-ray device as claimed in claim 4, wherein the electric motor is disposed on an adjustable holder, and the rack is disposed on the compression plate or on a component connected to the compression plate.

6. The mammography x-ray device as claimed in claim 5, wherein the rack is disposed on a guide rail connected to the compression plate, the compression plate being laterally displaceable in relation to the adjustable holder.

7. The mammography x-ray device as claimed in claim 6, wherein the guide rail is supported on the adjustable holder using a sliding guide.

8. The mammography x-ray device as claimed in claim 5, wherein the compression apparatus further comprises at least one sensor device, the at least one sensor device operable to detect a position of the compression plate relative to the adjustable holder.

9. The mammography x-ray device as claimed in claim 8, wherein the at least one sensor device comprises at least one light barrier, the at least one light barrier operable to detect the position of the compression plate.

10. The mammography x-ray device as claimed in claim 8, wherein the mammography x-ray device further comprises a control system or is coupled to a control system, the control system being configured to control the drive device based on a signal delivered by the at least one sensor device.

11. The mammography x-ray device as claimed in claim 2, wherein the drive device comprises an electric motor.

12. The mammography x-ray device as claimed in claim 3, wherein the electric motor is a geared motor.

13. The mammography x-ray device as claimed in claim 12, wherein the electric motor has a pinion that engages with a rack.

14. The mammography x-ray device as claimed in claim 6, wherein the compression apparatus further comprises at least one sensor device, the at least one sensor device operable to detect a position of the compression plate relative to the adjustable holder.

15. The mammography x-ray device as claimed in claim 7, wherein the compression apparatus further comprises at least one sensor device, the at least one sensor device operable to detect a position of the compression plate relative to the adjustable holder.

16. The mammography x-ray device as claimed in claim 9, wherein the at least one sensor device is a forked light barrier.

17. The mammography x-ray device as claimed in claim 9, wherein the at least one light barrier is operable to detect the position of the compression plate, such that recesses in a component connected to the compression plate are detected.

18. The mammography x-ray device as claimed in claim 9, wherein the mammography x-ray device further comprises a control system or is coupled to a control system, the control system being configured to control the drive device based on a signal delivered by the at least one sensor device.
